(19) United States
(12) Patent Application Publication
(10) Pub. No.: US 2026/0165775 A1
(43) Pub. Date: Jun. 18, 2026

(54) CATHETER SHAFT CONSTRUCTION WITH EXPOSED BRAID AS ELECTRODE(S)

(71) Applicant: Medtronic, Inc., Minneapolis, MN (US)

(72) Inventors: Gavin J. Kenny, Galway (IE); Brian J. Kelly, Oranmore (IE); Brian T. Howard, Minneapolis, MN (US); Mark T. Stewart, Lino Lakes, MN (US); Timothy G. Laske, Shoreview, MN (US); Andrzej M. Malewicz, Minnetonka, MN (US); Jesse J. Pischlar, Lino Lakes, MN (US)

(21) Appl. No.: 19/123,645

(22) PCT Filed: Oct. 26, 2023
(86) PCT No.: PCT/US2023/077926

§ 371 Date: Apr. 23, 2025

Related U.S. Application Data
(60) Provisional application No. 63/381,224, filed on Oct. 27, 2022.

(51) Int. Cl. A61B 18/14; A61B 18/00

(52) U.S. Cl. CPC A61B 18/1492; A61B 2018/00077; A61B 2018/00083; A61B 2018/00136; A61B 2018/0016; A61B 2018/0022; A61B 2018/00351; A61B 2018/00577; A61B 2018/1407; A61B 2018/1417; A61B 2018/1467

(57) ABSTRACT

An example ablation catheter includes an elongated structure configured to be at least partially inserted into an organ of a patient, the elongated structure comprising a metallic element, the metallic element comprising a plurality of electrically conductive metallic wires comprising a polymer coated portion and an exposed metallic portion. The ablation catheter also includes a plurality of electrodes disposed at a distal portion of the elongated structure. At least one of the plurality of electrodes and the exposed metallic portion are configured to provide a current path for pulsed field ablation.

This application claims the benefit of U.S. Provisional Patent Application No. 63/381,224, filed on Oct. 27, 2022, the entire content of which is incorporated herein by reference.

### TECHNICAL FIELD

The present technology is related to ablation catheters. In particular, various examples of the present technology are related a pulsed field ablation catheter having an element with exposed metallic braid or exposed metallic coils, which is configured to operate as one or more electrodes.

### BACKGROUND

Tissue ablation is a medical procedure commonly used to treat conditions such as cardiac arrhythmia, which includes atrial fibrillation. For treating cardiac arrhythmia, ablation can be performed to modify tissue, such as to stop aberrant electrical propagation and/or disrupt aberrant electrical conduction through cardiac tissue. Ablation techniques include pulsed field ablation (PFA), cryoablation, and radiofrequency (RF) ablation. In particular, RF or pulsed field ablation may be applied via electrodes of an ablation catheter.

Cardiac arrhythmias are a group of conditions that cause an irregular heartbeat. Ablation may be used to kill or isolate tissue responsible for causing arrythmias and thereby restore or improve heart function. Furthermore, optimized control of the applied therapy through specialized tools allows for therapeutic delivery that can be performed without causing damage to adjacent structures or surrounding tissue, ideally resulting in no need for a maintenance treatment regimen, such as medications or cardioversions.

### SUMMARY

The present technology is directed to devices for pulsed field ablation using an ablation catheter having a metallic element with exposed metallic braid, weave, or mesh, or exposed metallic coils which may function as one or more electrodes and techniques for manufacturing such devices. For example, an ablation catheter may include a shaft or elongated structure including a kink resistant element, or element to enhance torque transfer, such as a metallic braid (which may also be referred to as a metallic weave or metallic mesh) or a plurality of metallic coils which may add kink resistance and/or structural rigidity to the catheter for insertion into anatomy of a patient, such as a heart of the patient. The individual metallic elements may each include a polymeric insulative coating which may provide a level of insulation to prevent or limit the conduction of electricity between electrodes positioned on the surface of the catheter and the metallic braid or metallic coils. A polymer jacket coating will typically be applied which covers over all of the internal metallic elements. The polymer jacket coating may be partially or completely removed, such as through laser ablation, thermal ablation, or mechanical force, from one or more portions of the insulative coating protecting individual metallic braid or metallic coils to expose the metal of the selected metallic braid wire(s) or the metallic coils to the surrounding environment. In some examples, the exposed metal may form one or more electrodes. For example, the exposed metal may form one or more return electrodes. Such return electrodes may be coupled to ground or to an opposing polarity of a pulsed field ablation generator. Alternatively, or additionally, the exposed metal may be electrically coupled to one or more electrodes and therefore act as a conductor for the one or more electrodes, rather than act as an electrode.

In accordance with one or more aspects of this disclosure, an ablation catheter includes: an elongated structure configured to be at least partially inserted into a patient, the elongated structure comprising a metallic element, the metallic element comprising a plurality of electrically conductive metallic wires comprising a polymer coated portion and an exposed metallic portion; and a plurality of electrodes disposed at a distal portion of the elongated structure, wherein at least one of the plurality of electrodes and the exposed metallic portion are configured to provide a current path for at least one of pulsed field ablation or sensing.

In another example, a method includes: providing an elongated structure comprising a metallic element, the metallic element comprising a plurality of electrically conductive metallic wires having a polymer coating disposed thereon; removing at least a portion of the polymer coating from a portion of the plurality of electrically conductive metallic wires to create a polymer coated portion and an exposed metallic portion; and disposing a plurality of electrodes at a distal portion of the elongated structure, wherein at least one of the plurality of electrodes and the exposed metallic portion are configured to provide a current path for at least one of pulsed field ablation or sensing.

The details of one or more aspects of the disclosure are set forth in the accompanying drawings and the description below. Other features, objects, and advantages of the techniques described in this disclosure will be apparent from the description and drawings, and from the claims. It should be appreciated in this disclosure that the term “sensing” is used to refer to a range of applications, including but not limited to, measurement of cardiac electrical signals such as cardiomyocyte depolarization potentials, local electrical tissue impedance values, electrical voltage measurements of currents passed through the body for positioning and navigation purposes, and electromagnetically transmitted signals.

### BRIEF DESCRIPTION OF THE DRAWINGS

FIGS. 1A-1B are conceptual diagrams illustrating an example system for delivering ablation therapy in accordance with one or more aspects of this disclosure.

FIG. 2 is a block diagram illustrating an example controller of an ablation system, in accordance with one or more aspects of this disclosure.

FIGS. 3A-3C are conceptual diagrams illustrating examples of an ablation catheter having an expandable structure in accordance with one or more techniques of this disclosure.

FIGS. 4A-4D are conceptual diagrams illustrating an example catheter having a mechanical expandable structure in accordance with one or more techniques of this disclosure.

FIG. 5 is a conceptual diagram of a portion of another example catheter in accordance with one or more techniques of this disclosure.

FIGS. 6A-6E are conceptual diagrams illustrating a portion of an example metallic element in accordance with one or more aspects of this disclosure.

FIG. 7 is a conceptual diagram illustrating an example metallic element in accordance with one or more aspects of this disclosure.

FIG. 8 is a flowchart illustrating techniques for manufacturing an ablation catheter in accordance with one or more aspects of this disclosure.

### DETAILED DESCRIPTION

An ablation catheter may include an elongated structure or shaft having a metallic element such as a metallic braid (which may also be referred to as a metallic weave or metallic mesh) or a plurality of metallic coils, which may provide a level of kink resistance, effective torque transfer, and flexibility to the catheter, such that the catheter does not kink up when being inserted into or advanced in anatomy of a patient. This metallic element may include a polymer coating to provide for insulation to or within the metallic element. However, even with a polymer coating, such a metallic element may act to short out an electric field being created by electrodes of the ablation catheter, particularly when the ablation catheter is operating at relatively high voltages. Additionally, in constructing such a catheter, crimping, swaging, or welding an electrode onto the surface of the catheter may cause the electrode to short to the underlying metallic element.

Techniques are disclosed herein that take advantage of the existence of such a metallic element in an ablation catheter. For example, a portion of the metallic element may have the polymer coating partially or completely removed so as to expose that portion of the metallic element to a surrounding environment. The portion of the metallic element that has the polymer coating partially or completely removed may be referred to herein as an “exposed metallic portion.” The exposed metallic portion of the metallic element may be used as an electrode. For example, the exposed metallic portion may be used as a return electrode for current being used to ablate tissue or for sensing. By using the exposed metallic portion as a return electrode, current may flow safely from inside the patient to a pulse generator. Alternatively, an electrode may be attached to the exposed metallic portion of the metallic element, and the exposed metallic portion of the metallic element may function as a conductor for the electrode.

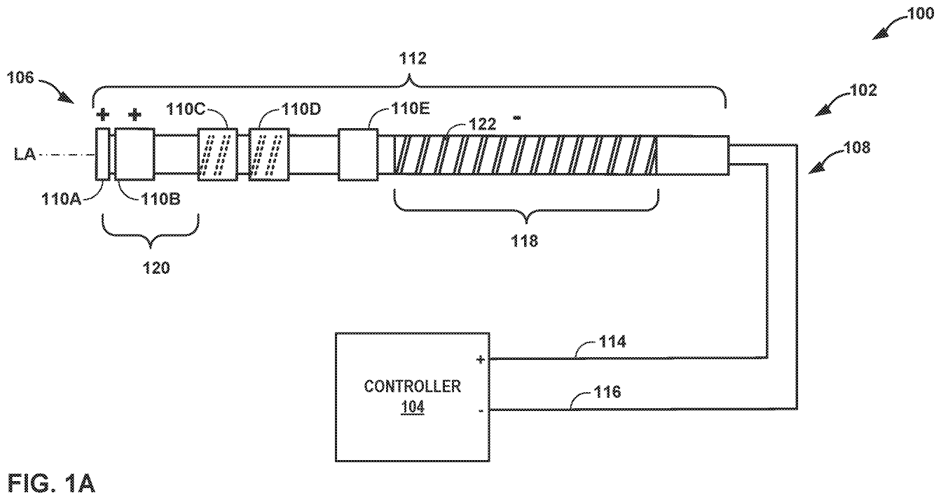

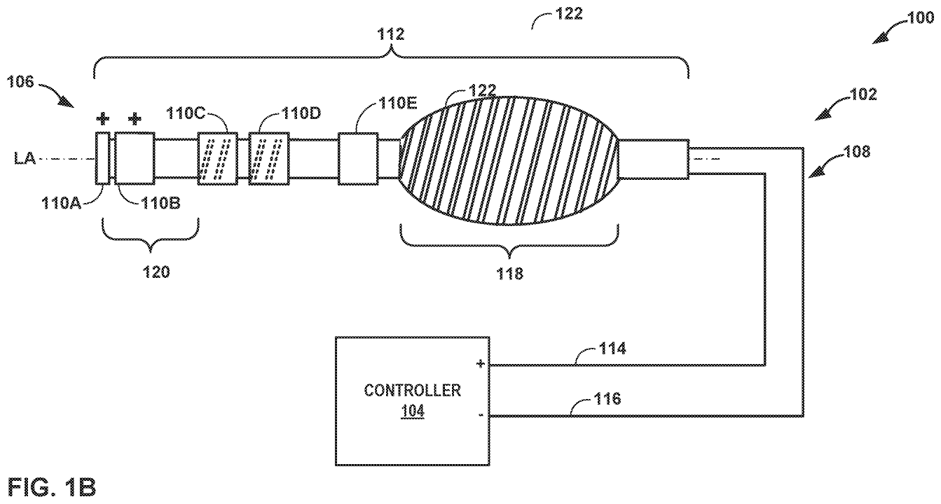

FIGS. 1A-1B are conceptual diagrams illustrating an example system for delivering ablation therapy in accordance with one or more aspects of this disclosure. System 100 includes a catheter 102, and a controller 104. In general, to deliver ablation, a practitioner (e.g., cardiologist, surgeon, etc.) may insert one or more of catheter 102 into a patient and cause controller 104 to deliver, via catheter 102, energy (e.g., pulsed field ablation energy) to target tissue of a patient. In a cardiac patient, ablation may cause lesions in target tissue of a heart of a patient which may mitigate or stop cardiac arrhythmia. As ablation is generally intended to cause lesions in the heart, it may be desirable to control the ablation catheter to safely provide a return path to a pulse generator for current being used for the ablation.

Catheter 102 may include elongated structure 112 including a plurality of electrodes 110A-110E (collectively “electrodes 110”). While the example of FIG. I includes five such electrodes, catheter 102 may include any number of electrodes. In some examples, each of electrodes 110 may be configured to be selectably be used as a cathode or an anode. As such, an electric field may be generated inside anatomy of a patient between one or more cathodes and one or more anodes, in a bi-polar manner.

Elongated structure 112 may include a metallic element which may provide some level of kink resistance and/or effective torque transfer to catheter 102. The metallic element may include a polymer coated portion 120 and an exposed metallic portion 118. For example, exposed metallic portion 118 may originally include a polymer coating, which may be partially or completely removed, for example, by laser ablation, thermal ablation, or by mechanical forces. Exposed metallic portion 118 may form a thermocouple and be configured to operate as an electrode, such as a return electrode, during delivery of pulsed field ablation therapy. For example, one or more of electrodes 110 may be electrically coupled to a first terminal of controller 104 (e.g., via conductor 114) and exposed metallic portion 118 may be electrically coupled to a second terminal of controller 104 (e.g., via conductor 116) having an opposite polarity than the first terminal. In the example of FIG. 1, the first terminal is positive and the second terminal is negative. It should be understood that the first terminal may be negative and the second terminal may be positive or the first terminal and the second terminal may alternate polarities such that when one terminal is positive, the other terminal is negative. In some examples, rather than being coupled to a second terminal of controller 104, exposed metallic portion 118 may be electrically coupled to ground. In some examples, one or more of electrodes 110 may be electrically coupled to the second terminal of controller 104 or to ground, as well. Conductors 114 and 116 may run through a handle of catheter 102 (not shown) so as to be readily connectible to controller 104.

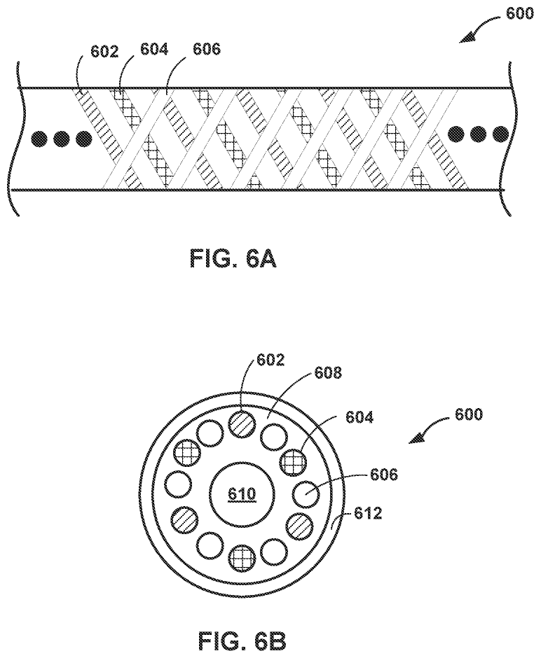

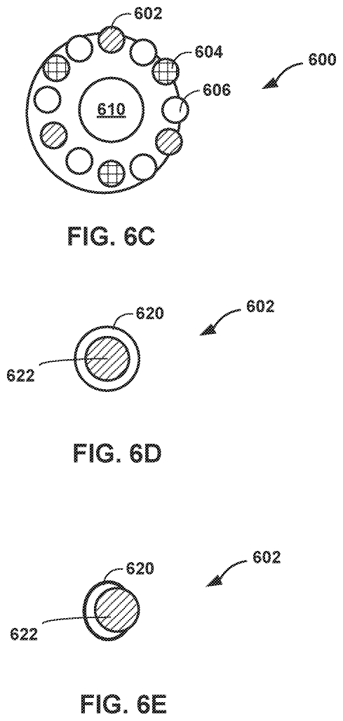

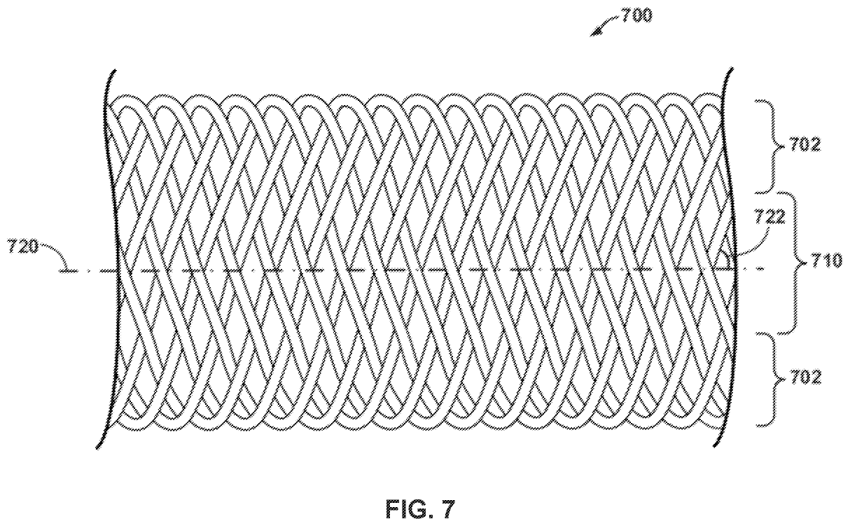

In some examples, the metallic element may include a plurality of electrically conductive metallic wires running together side by side, such as a tight corkscrew. In some examples, the metallic element may include electrically conductive metallic wires that crisscross each other as a braid. In some examples, polymer filaments may be woven in with the plurality of wires in the metallic element of elongated structure 112. A further discussion of an example metallic element follows later in this disclosure with respect to FIGS. 6A-6E.

A manufacturer of such an ablation catheter may laser ablate, thermal ablate, or mechanically remove away the polymer coating from metallic element to make a portion of the metallic element into an electrode. In some examples, the manufacturer may employ a computer vision system to assist with ablating the polymer coating from the metallic element in order to more precisely remove only the desired amount of the polymer coating in the desired location. By moving the location of the ablation, different electric fields for ablation may be generated. In this manner, a manufacturer may relatively easily manufacture ablation cathodes with different ablation properties.

Exposed metallic portion 118 may be used as an electrode when delivering pulsed field ablation therapy. In some examples, exposed metallic portion 118 may include a plurality of exposed electrically conductive metallic wires, such as exposed metallic wire 122. The plurality of exposed electrically conductive metallic wires may provide built in redundancy as each of the plurality of exposed electrically conductive metallic wires may be electrically coupled together due to the removal of the polymer coating. For example, exposed metallic portion 118 may be coupled to a negative terminal of controller 104 via conductor 116. Conductor 116 may be electrically coupled to each of the plurality of electrically conductive metallic wires in exposed metallic portion 118 such that if one of the plurality of electrically conductive metallic wires breaks, conductor 116 may continue to provide electrical connectivity to the unbroken electrically conductive metallic wires of the plurality of electrically conductive metallic wires in exposed metallic portion 118. Alternatively, or additionally, an additional one or more of the plurality of electrically conductive metal wires in exposed metallic portion 118 may have independent conductor connections back to the second terminal or a similarly polarized terminal of controller 104. In some examples, each wire of the plurality of electrically conductive metallic wires of exposed metallic portion 118 may function as a separate electrode. For example, the polymer coating between the different wires of the plurality of electrically conductive metallic wires may not be removed and only the polymer coating on an outer surface or a portion of an outer surface of one or more of the plurality of electrically conductive metallic wires may be removed, thereby maintaining insulation between the wires, but exposing the metal of the wires such that the wires may individually be usable as electrodes. Exposed metallic portion 118 may be on the order of 10-30 millimeters long and be located several millimeters proximal of an ablation electrode, such as electrodes 110. In some examples, after creating exposed metallic portion 118, a manufacturer may apply an insulative coating to a portion of exposed metallic portion 118 to facilitate focusing an electrical field in a desired manner.

In some examples, a portion of elongated structure 112 may have the polymer coating on the metallic element partially or completely removed and an electrode may be affixed to the metallic element over the exposed metal of the metallic element. For example, the polymer coating on the electrically conductive metallic wires of the metallic element may be partially or completely removed at the location of electrode 110C and electrode 110D as shown in FIG. 1 (represented with dotted lines). Electrodes 110C and 110D may then be crimped, swaged, welded (e.g., laser welded), or otherwise affixed onto the exposed metal.

As can be seen, catheter 102 includes electrodes 110 on a distal portion of elongated structure 112. In the example of FIG. 1, electrodes 110A and 110B are positively charged using controller 104. Elongated structure 112 may include a metallic element, which may include polymer filaments and metallic coils (e.g., electrically conductive metallic wires) or metallic braid which may include electrically conductive metallic wires. Exposed metallic portion 118 has the polymer coating removed, such as by laser ablation, thermal ablation, or mechanical force, to leave the at least a portion of metallic coils or metallic braid exposed to the surrounding environment. In some examples, elongated structure 112 may be mechanically coupled to a lever in a handle (not shown) which allows the position of exposed metallic portion 118 to be adjusted relative to the distal fixed electrodes and/or the density of exposed metallic portion 118 to be adjusted. For example, the lever may compact the coils or braid together or stretch the coils or braid apart. Exposed metallic portion 118 may be connected to a negative terminal of controller 104 via conductor 116. The application of voltage between electrodes (e.g., electrodes 110A and electrode 110B) allows for a current to be created between the electrodes and exposed metallic portion 118 of elongated structure 112, thus creating an electric field. The electric field may ablate target tissue of the anatomy of the patient. In some examples, the intensity of the electrical field may be adjusted by moving the position of exposed metallic portion, for example by the lever in the handle.

Catheter 102 may generally include features that enable insertion of catheter 102 into a patient and navigation of catheter 102 to a target tissue site. In some examples, catheter 102 may include a molded polymer insert configured to reduce or eliminate sharp edges to ease insertion and advancement of catheter 102 into anatomy of a patient. Elongated structure 112 may include a distal portion 106 and a proximal portion 108. Electrodes 110 may be generally positioned at distal portion 106, while proximal portion 108 may be connected to controller 104. Electrodes 110 may be of any suitable geometry. Example geometries of electrodes 110 include, but are not necessarily limited to, circular (e.g., ring) electrodes surrounding the body of the lead, C-shaped electrodes which partially surround the body of the lead, other curved shaped electrodes, pigtail shaped electrodes, spiral shaped electrodes, conformable electrodes, cuff electrodes, segmented electrodes (e.g., electrodes disposed at different circumferential positions around the lead instead of a continuous ring electrode), any combination thereof (e.g., ring electrodes and segmented electrodes). In some examples, where one or more of electrodes 110 are spiral shaped, the one or more of electrodes 110 may be a fixed pitch spiral shape, a variable pitch spiral shape, or a variable diameter spiral shape. Electrodes 110 may be made of nitinol, copper, stainless steel, or other conductive metallic material. In some examples, to better deliver therapeutic high voltage pulses, electrodes 110 may be clad or coated with a suitable metal surface, such as platinum, platinum-iridium alloy, tantalum, gold, nitrides (e.g., titanium nitride, tantalum nitride, palladium nitride, or rhodium nitride), and/or the like. In some examples, electrodes 110 may be laser cut from a single metal tube.

Electrodes 110 may be axially distributed along longitudinal axis LA of elongated structure 112. Elongated structure 112 may include conductors, such as conductor 114 and conductor 116 configured to carry electrical signals between electrodes 110 and controller 104. In some examples, elongated structure 112 may include a separate conductor for each of electrodes 110 and for exposed metallic portion 118. For instance, in the example of FIG. 1 where electrodes 110 includes five electrodes 110 and exposed metallic portion 118, elongated structure 112 may include six separate conductors. In this way, elongated structure 112 may enable each electrode of electrodes 110 and exposed metallic portion 118 to be driven with a different signal from controller 104. In other examples, multiple electrodes of electrodes 110 and/or exposed metallic portion 118 may share a common conductor. For instance, electrodes 110C and 110D may be connected to a same (e.g., a common) conductor. While such a common conductor arrangement may reduce electrode flexibility (e.g., as electrodes connected to the common conductor may be driven with a same signal), such an arrangement may reduce manufacturing complexity and/or cost, and may increase the structural flexibility of catheter 102.

As shown in FIG. 1A, electrodes 110 may include a tip electrode (e.g., electrode 110A), which may be a ring electrode with a “cap” covering at least a portion of a tip of elongated structure 112. In some examples, the tip electrode may be chamfered or otherwise rounded (e.g., to enable easier passage of catheter 102 through anatomy of the patient). Electrodes 110 may include a tip ring electrode (e.g., electrode 110B) that is adjacent to the tip electrode. The tip ring electrode may be separated (axially along LA) from the tip electrode. Electrodes 110 may include one or more pairs of ring electrodes. A pair of ring electrodes may include two adjacently closely spaced electrodes of electrodes 110. For instance, in the example of FIG. 1A, electrodes 110C and 110D may form a first pair of ring electrodes. In general, the first pair of ring electrodes (i.e., electrodes 110C and 110D) may be accompanied by one or more additional electrodes. The one or more additional electrodes may include any combination of pairs of ring electrodes, coil electrodes (e.g., electrodes that include conductors that spiral around elongated structure 112) or other shaped electrodes.

In the example of FIG. 1A, electrodes 110 are illustrated as having a larger diameter than elongated structure 112. In some examples, one or more of electrodes 110 may have a diameter that is approximately equal to a diameter of elongated structure 112. For instance, electrodes 110 may be recessed in elongated structure 112 such that the combination results in a relatively smooth outer surface.

Controller 104 may include an energy generator configured to provide electrical pulses to electrodes 110 via conductors, such as conductor 114 and conductor 116, to perform an ablation procedure to cardiac tissue or other tissues within the patient's body, such as renal tissue, airway tissue, and organs or tissue within the cardiac space or the pericardial space. For instance, the energy generator may be configured and programmed to deliver pulsed, high-voltage electric fields appropriate for achieving desired pulsed, high-voltage ablation (referred to as “pulsed field ablation” or “pulsed electric field ablation”).

FIG. 1B depicts catheter 102 in a compacted position. For example, elongated structure 112 may be mechanically coupled to a lever in a handle (not shown) which allows the position of exposed metallic portion 118 to be adjusted relative to the distal fixed electrodes and/or the density of exposed metallic portion 118 to be adjusted. For example, the lever may compact the electrically conductive metallic wires of exposed metallic portion 118 together or stretch the electrically conductive metallic wires of exposed metallic portion apart. In this example, the electrically conductive metallic wires of exposed metallic portion 118 have been compacted together. For example, the distance between electrically conductive metallic wires, such as exposed metallic wire 122 and other wires, of exposed metallic portion 118 may be closer together in FIG. 1B than in FIG. 1A along longitudinal axis LA of elongated structure 112.

In accordance with the techniques of this disclosure, an ablation catheter comprises an elongated structure being configured to be at least partially inserted into an organ of a patient, the elongated structure comprising a metallic element, the metallic element comprising a metallic braid or a plurality of metallic coils, the metallic braid or plurality of metallic coils comprising a plurality of electrically conductive metallic wires, the plurality of electrically conductive metallic wires comprising a polymer coated portion and an exposed metallic portion, wherein the exposed metallic portion is configured to provide a return path for electricity to a pulsed field ablation generator; and a plurality of electrodes disposed at a distal portion of the elongated structure.

In accordance with the techniques of this disclosure, a method comprises providing an elongated structure comprising a metallic element, the metallic element comprising a metallic braid or a plurality of metallic coils, the metallic braid or plurality of metallic coils comprising a plurality of electrically conductive metallic wires having a polymer coating disposed thereon; removing at least a portion of the polymer coating from a portion of the plurality of electrically conductive metallic wires to create a polymer coated portion and an exposed metallic portion, wherein the exposed metallic portion is configured to provide a return path for electricity to a pulsed field ablation generator; and disposing a plurality of electrodes at a distal portion of the elongated structure.

In some examples, one or more exposed metallic portions of elongated structure 112 may be used as sense electrodes to sense an impedance of a body to determine a location of elongated structure 112 within a body of a patient. For example, one or more exposed metallic portions of system 100 may function as electrodes such that controller 104 may sense a voltage level of one or multiple electrical currents being transmitted through the body cavity from one or more body surface patch electrodes (also not shown) to the one or more exposed metallic portions. For example, a plurality of constant current signals (e.g., three) may be driven through the body of the patient via a plurality of patch electrodes which may be deployed in orthogonal pairs (e.g., a total of six patch electrodes). The sensing elements may sense a voltage drop as a function of position within the body because the impedance of the body tissue and blood influences the voltage measurements of the sensing elements. Controller 104 may then use such sensed voltage levels to determine a position of the sensing elements (e.g., the exposed metallic portions) and associated device structure in the body of the patient.

In some examples, system 100 may sense transmitted magnetic fields to determine positioning of elongated structure 112 within the body, for example, when exposed metallic portion 118 includes at least one coil. In some examples, a plurality of exposed metallic portions, like exposed metallic portion 118, may function as sensors for determining position(s) in the body, For example, elongated structure 112 may include a plurality of exposed metallic portions, such as at a distal end of elongated structure 112 as well as at other locations along elongated structure 112. The sensor(s) may sense currents from such exposed metallic portions to determine a position of elongated structure 112 in the body.

Although not shown, system 100 may include one or more sensors to monitor the operating parameters through the medical system 100, such as temperature, delivered voltage, or the like, and for measuring and monitoring one or more tissue characteristics, such as electrogram waveforms, monophasic action potentials, tissue impedance, or the like, in addition to monitoring, recording, or otherwise conveying measurements or conditions within the energy delivery device or other component of system 100 or the ambient environment at the distal portion of the energy delivery device. The sensor(s) may be in communication with controller 104 for initiating or triggering one or more alerts or ablation energy delivery modifications during operation of the energy delivery device. In some examples, such sensors may be part of controller 104.

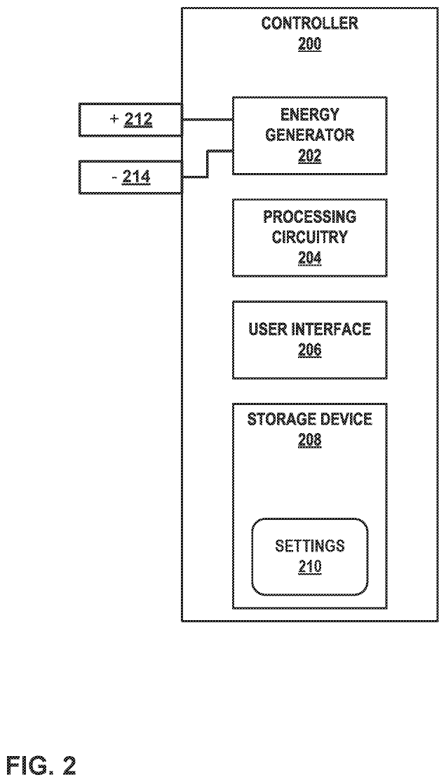

FIG. 2 is a block diagram illustrating an example controller of an ablation system, in accordance with one or more aspects of this disclosure. Controller 200 of FIG. 2 may be an example of controller 104 of FIG. 1. As shown in FIG. 2, controller 200 may include positive terminal (+) 212, negative terminal (−) 214, energy generator 202, processing circuitry 204, user interface 206, and storage device 208.

Positive terminal 212 may be coupled to energy generator 202 and may be configured to attach to conductor 114 (FIG. 1) so as to conduct electricity between energy generator 202 and conductor 114. Negative terminal 214 may be coupled to energy generator 202 (or alternatively to ground) and may be configured to attach to conductor 116 (FIG. 1) so as to conduct electricity between conductor 116 and energy generator 202. Energy generator 202 may be configured to control electrodes 110 and/or exposed metallic portion 118 of catheter 102 (FIG. 1) such as to provide electrical pulses to electrodes (e.g., electrodes 110) to perform an electroporation procedure or other ablation procedure to cardiac tissue or other tissues within the patient's body, such as renal tissue, airway tissue, and organs or tissue within the cardiac space or the pericardial space. For instance, energy generator 202 may be configured and programmed to deliver pulsed, high-voltage electric fields appropriate for achieving desired pulsed, high-voltage ablation (referred to as “pulsed field ablation” or “pulsed electric field ablation”). While shown in the example of FIG. 2 as a single energy generator, energy generator 202 is not so limited. For instance, controller 200 may include multiple energy generators that are each capable of generating ablation signals in parallel. In some examples, controller 200 may include energy generators of different types, such as a pulsed field energy generator, a radio frequency energy generator, and/or a cryogenic energy generator. In some examples, the cryogenic energy generator may be part of catheter 102 and be controlled electrically by processing circuitry 204.

Processing circuitry 204 may include one or more processors, such as any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 204 herein may be embodied as firmware, hardware, software or any combination thereof. Processing circuitry 204 controls energy generator 202 to generate signals according to various settings 210 which may be stored in storage device 208.

Storage device 208 may be configured to store information within controller 200, respectively, during operation. Storage device 208 may include a computer-readable storage medium or computer-readable storage device. In some examples, storage device 208 includes one or more of a short-term memory or a long-term memory. Storage device 208 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 208 is used to store data indicative of instructions, e.g., for execution by processing circuitry 204, respectively.

User interface 206 may include a button or keypad, lights, a speaker/microphone for voice commands, and/or a display, such as a liquid crystal (LCD), light-emitting diode (LED), or organic light-emitting diode (OLED). User interface 206 may be configured to receive input from a clinician, such as selecting settings from settings 210 for use during an ablation therapy session. In some examples, the display may be configured to display information regarding an in-progress ablation therapy session, such as patient parameters or other information which may be useful to a clinician.

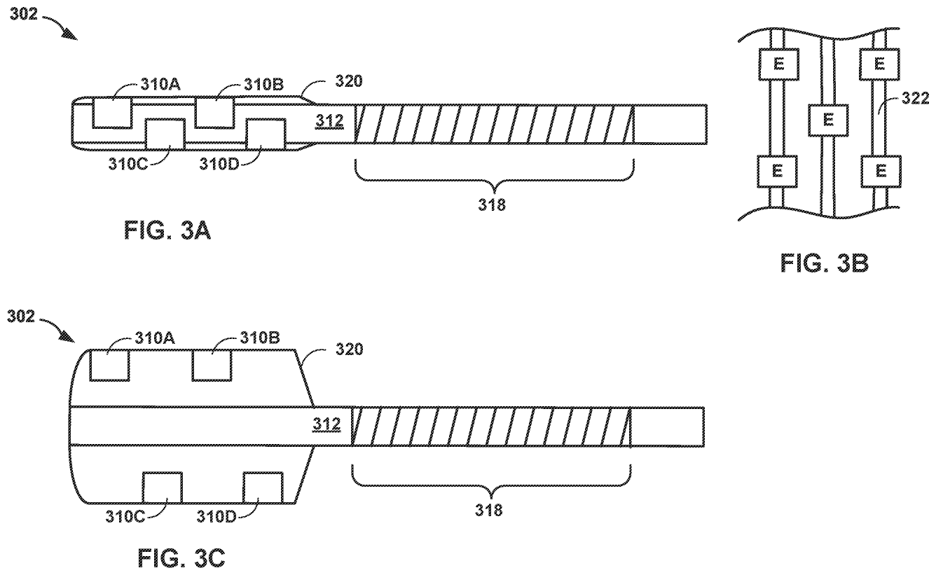

FIGS. 3A-3C are conceptual diagrams illustrating examples of an ablation catheter having an expandable structure in accordance with one or more techniques of this disclosure. FIG. 3A depicts an example catheter 302 having expandable structure 320 in an unexpanded (or contracted) state. In an unexpanded state, catheter 302 may be easier to insert into the anatomy of the patient than with expandable structure 320 in an expanded state. For example, expandable structure 320 may include a balloon. The balloon may be filled by controller 104 or a pump with a gas or liquid via tubing (not shown). Electrodes 310A, 310B, 310C, and 310D (collectively “electrodes 310”) are shown affixed to an outer surface of expandable structure 320. By expanding expandable structure 320, a clinician may locate one or more of electrodes 310 at an appropriate location and distance from target tissue within the anatomy of the patient to perform ablation therapy on the target tissue, As can be seen, electrodes 310 are offset from each other on the surface of expandable structure 320 so as to minimize a size of catheter 302 when expandable structure 320 is in an unexpanded state. Exposed metallic portion 318 may represent an example of exposed metallic portion 118 of FIG. 1 and may be used as an electrode, such as a return electrode.

FIG. 3B depicts a different view of electrodes (labeled E) in an offset position which may be examples of electrodes 310. The electrodes of FIG. 3B may be coupled to conductors, such as conductor 322, which may be configured to conduct electricity to or from controller 104 of FIG. 1.

FIG. 3C depicts catheter 302 with expandable structure 320 in an expanded position. As can be seen, when expandable structure 320 is in an expanded state, electrodes 310 are further away from expandable structure 312 than when in an unexpanded state (FIG. 3A), and more likely, when in an anatomy of a patient, to be closer to tissue of the patient.

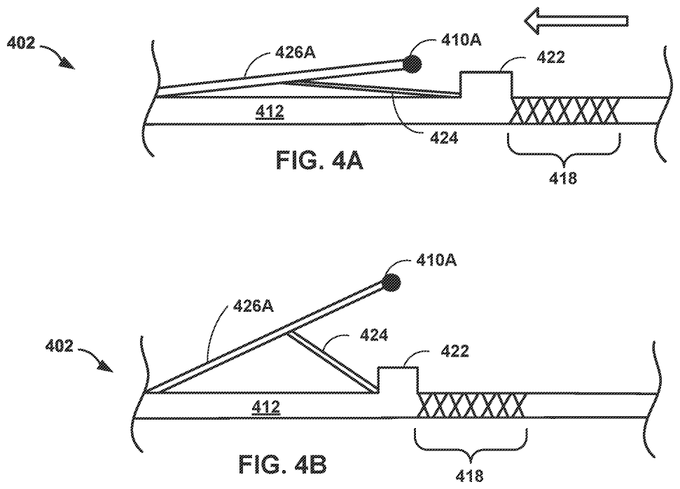

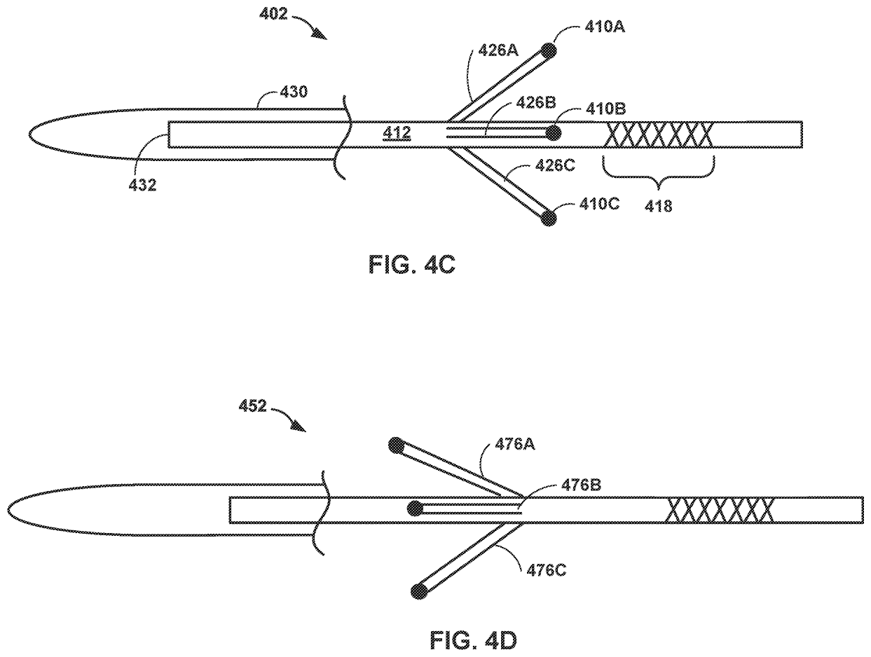

FIGS. 4A-4D are conceptual diagrams illustrating an example catheter having an expandable mechanical structure in accordance with one or more techniques of this disclosure. In the examples of FIGS. 4A-4C, catheter 402 may include an expandable mechanical structure that may be similar in appearance to a cocktail umbrella. The expandable mechanical structure may be configured to be manipulated from a neutral or axial configuration to an erect or semi-erect configuration by a mechanical device, lever, or the like, which may be accessed via a handle (not shown) of catheter 402. In some examples, catheter 402 may include a core (not shown in FIGS. 4A-4C) which may run axially through catheter 402 providing some stiffening properties to facilitate the advancing of catheter 402 through anatomy of a patient. Such expandable mechanical structure may be made of any suitable material, such as nitinol.

Catheter 402 may include a plurality of electrodes, such as electrode 410A. In the example of FIGS. 4A-4C, the plurality of electrodes may be generally spherical in shape and may be welded, crimped, swaged, formed on, or otherwise coupled to electrode arms, such as electrode arm 426A. Catheter 402 also may include exposed metallic portion 418 which may be an example of exposed metallic portion 118 (FIG. 1) or exposed metallic portion 318 (FIG. 3). FIGS. 4A and 4B depict an example of one electrode arm of such a mechanical structure and a deployment mechanism for such an electrode arm. In the example of FIG. 4A, exposed metallic portion 418 is shown as having electrically conductive metallic wires crisscrossing each other rather than spiraled electrically conductive metallic wires of exposed metallic portion 118 or exposed metallic portion 318. Electrode 410A is disposed upon electrode arm 426A. Electrode arm 426A is shown in a semi collapsed or unexpanded state. In some examples, when electrode arm 426A is in a completely collapsed or unexpanded state, electrode arm 426A may be relatively parallel to the body of catheter 402 so as to maintain a relatively low profile when in the collapsed or unexpanded state. Electrode arm 426A may be mechanically coupled or in contact with deployment arm 424. Deployment arm 424 may be mechanically coupled to or in contact with outcropping 422. Outcropping 422 may be moveable by a clinician through a mechanical device or lever via a handle of catheter 402 (not shown). For example, outcropping may be coupled to a mechanical deployment device. By moving outcropping 422 in the direction indicated by the arrow shown in FIG. 4A, outcropping 422 pushes against deployment arm 424 which causes electrode arm to be deployed, moving electrode 410A away from the body of catheter 402 as shown in FIG. 4B.

FIG. 4C depicts a view of catheter 402 in an expanded state. In the example of FIG. 4C, catheter 402 may include a plurality of electrodes such as electrode 410A, 410B, and 410C (collectively “electrodes 410”). Each of electrodes 410 may be disposed on a respective electrode arm such as electrode arms 426A, 426B, and 426C (collectively “electrode arms 426”). While not depicted in FIG. 4C, another electrode arm may be located on an opposite side of electrode arm 426B having an electrode disposed thereon. In some examples, there may be more or less electrode arms having electrodes disposed thereon than depicted in FIG. 4C. Each of electrode arms 426 may be coupled to or in contact with a respective deployment arm (not shown in FIG. 4C), such as deployment arm 424 of FIGS. 4A-4B. Exposed metallic portion 418 is shown and may be configured to be an electrode, such as a return electrode.

In some examples, catheter 402 may include a sheath 430. Sheath 430 may be configured make it easier to insert and advance catheter 402 into anatomy of a patient and to protect the patient during insertion and advancement of catheter 402 into the anatomy of the patient. For example, sheath 430 may cover a distal end 432 of elongated structure 412. In some examples, sheath 430 may cover one or more electrodes, such as electrodes 410 or other electrodes (not shown) during insertion and advancement of catheter 402 into the anatomy of the patient. In such examples, sheath 430 may be coupled to a mechanical device or lever within the handle of catheter 402 that a clinician may move to push sheath 430 distally to uncover the electrodes once the clinician has navigated catheter 402 to a desired position with the anatomy of the patient. Once the clinician delivers the ablation therapy via catheter 402, the clinician may utilize the mechanical device or lever to return the position of sheath 430 to covering the electrodes.

In some examples, rather than include deployment arms and outcroppings, electrode arms 426 may be biased to be in an expanded state and sheath 430 may cover electrode arms 426 when catheter 402 is in the unexpanded state, keeping electrode arms 426 near elongated structure 412 of catheter 402. Sheath 430 may be coupled to a mechanical device or lever within the handle of catheter 402 that a clinician may move to push sheath 430 distally to deploy the electrode arms into the expanded state. When an ablation procedure is complete, the clinician may retract the sheath proximally to re-cover electrode arms 426 and bring catheter 402 back into an unexpanded state via the mechanical device or lever. In some examples, the expandable mechanical structure is non-isodiametric.

FIG. 4D depicts a view of a catheter 452 in an expanded state. Catheter 452 may be similar to catheter 402 of FIGS. 4A-4C except that electrode arms 476A, 476B, and 476C (collectively “electrode arms 476”) may be hinged in a different direction than electrode arms 426 of FIGS. 4A-4C. In this manner, removal of catheter 452 from the patient may be performed more easily in the event of a malfunction of a deployment and/or retraction apparatus of the catheter.

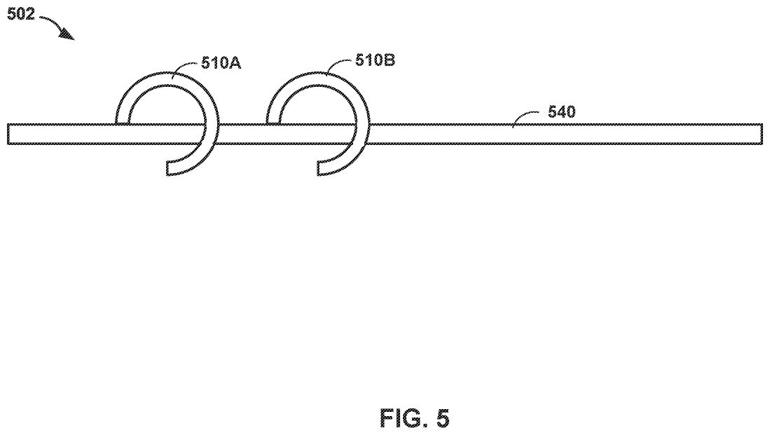

FIG. 5 is a conceptual diagram of a portion of another example catheter in accordance with one or more techniques of this disclosure. Catheter 502 may include a plurality of electrodes, such as electrodes 510A and 510B (collectively “electrodes 510”). Electrodes 510 may be curved in shape such as to resemble the letter “C”. While electrodes 510 are shown as curved shaped, alternatively electrodes 510 may be pigtail shaped or spiral shaped. In some examples, where one or more of electrodes 510 is spiral shaped, the one or more of electrodes 510 may be a fixed pitch spiral shape, a variable pitch spiral shape, or a variable diameter spiral shape. Each of electrodes 510 may partially surround an expandable structure (not shown) of catheter 502. For example, electrodes 510 may be laser cut from a single metal tube. Such a metal tube may include nitinol, copper, stainless steel, or another electrically conductive material. Conductor 540 may be similarly cut from the same single metal tube and be electrically coupled to electrodes 510. In some examples, conductor 540 may take the form of a ribbon. By cutting electrodes 510 and conductor 540 from a single metal tube, catheter 502 may have an increased structural rigidity and thereby facilitate the introduction and advancement of catheter 502 into the anatomy of the patient, compared to if the electrodes and conductor where individually manufactured and then coupled together. This increase structural rigidity may also help maintain spacing of the electrodes when catheter 502 is introduced and advanced in the anatomy of the patient.

For example, a manufacture may cut a series of slotted C shaped electrodes (e.g., electrodes 510) connected by a ribbon (e.g., conductor 540) from a single metal tube. Such a metal tube may be a nitinol tube, a copper tube, or another metal or alloy tube. In some examples, after cutting electrodes 510 and conductor 540 from the single metal tube, conductor 540 may be coated with a polymer coating. In some examples, the polymer coating may include any of polyimide, nylon, PTFE (polytetrafluoroethylene), FEP (fluorinated ethylene propylene), ETFE (ethylene tetrafluoroethylene), PVDF (polyvinylidene difluoride), polyurethane-nylon, crosslinked high-density polyethylene or polypropylene, Soluble Imide (SI) polyimide (formerly known as Genymer, Genymer SI), LaRC-SI, Pyre-ML polyimides, and/or the like. In some examples, the polymer coating may be formed of a hydrolytically stable polyimide, such as SI polyimide, or other relatively high dielectric strength polymer, to insulate conductor and allow for an electric field to be generated by electrodes 510. It should be noted that because electrodes 510 and conductor 540 may be cut from a same metal tube prior to coating conductor 540, electrical conductivity may be maintained between electrodes 510 and conductor 540. The ribbon wire provides for structural rigidity to maintain the spacing of the electrodes when an external load is applied due to movement of the catheter in the body.

FIGS. 6A-6E are conceptual diagrams illustrating a portion of an example metallic element in accordance with one or more aspects of this disclosure. FIG. 6A depicts a longitudinal or axial view of metallic element 600. Metallic element 600 may be an example of the metallic element of FIG. 1. Metallic element 600 may include one or more electrically conductive metallic wires, such as wire 602, which may include a conductive material, such as copper or copper alloy. Wire 602 may originally include a polymer coating which may surround the diameter of wire 602 along at least a portion of a length of wire 602 to provide electrical insulation to wire 602. For example, the polymer coating may include one or more materials such as those discussed above. Each of the electrically conductive metallic wires shown in FIG. 6A having a diagonal fill may be similar to wire 602 or may be part of wire 602, as wire 602 may wind around a core element (not shown in FIG. 6A) or be suspended in a substrate (not shown in FIG. 6A) in a spiral fashion. In some examples, the substrate may function as the polymer coating of wire 602. At least a portion of the polymer coating and/or substrate may be removed, via laser ablation, thermal ablation, or mechanical force, to expose at least a portion of the conductive material of wire 602 to the surrounding environment.

Wire 604 may include a same conductive material as wire 602 or a different conductive material than wire 602. For example, wire 604 may include constantan. Wire 604 may originally include a polymer coating which may surround the diameter of wire 604 along at least a portion of a length of wire 604 to provide electrical insulation to wire 604. For example, the polymer coating may include such as parylene N, polyethylene naphthalate (PEN), or other relatively high dielectric strength polymer. Each of the wires shown in FIG. 6A having a square or cross fill may be similar to wire 604 or may be part of wire 604, as wire 604 may wind around the core element or be suspended in a substrate in a spiral fashion. In some examples, the substrate may function as the polymer coating of wire 604. After laser ablating, at least a portion of the polymer coating and/or substrate may be ablated away to expose at least a portion of the conductive material of wire 604 to the surrounding environment. While shown as running substantially parallel to wire 602, in some examples, wire 604 may run in a different direction than wire 602. In some examples, wire 604 may form a braid or weave with wire 602 by running in a different direction and weaving behind and in front of wire 602.

Metallic element 600 may also include one or more polymer fibers, such as polymer fiber 606. Polymer fiber 606 may include a monofilament fiber Characteristics of polymer fiber 606 may include a relatively high tensile strength, a relatively high tensile modulus, a relatively high melt temp or softening temperature. In some examples, polymer fiber 606 may be include aromatic polyesters (such as “Vectran” or PEN), nylon, PEEK (polyetheretherketone), parylene N, or other relatively high tensile strength polymer. Each of the polymer fibers shown in FIG. 6A having no fill may be similar to polymer fiber 606 or may be part of polymer fiber 606, as polymer fiber 606 may wind around the core element or be suspended in the substrate in a spiral fashion. In some examples, polymer fiber 606 may wrap around the core or be suspended in the substrate in a different direction than wires 602 and 604. In some examples, polymer fiber 606 may form a braid or weave with wires 602 and 604 by weaving behind and in front of wires 602 and 604.

FIG. 6B, which may not be drawn to scale, depicts a cross section of metallic element 600 prior to removal of the polymer coating of at least a portion of metallic element 600. Metallic element 600 may include a core element 610, one or more electrically conductive metallic wires, such as wire 602 and wire 604, and one or more polymer fibers, such as polymer fiber 606. Core element 610 may include a hollow core or lumen through which mechanical control wires, electrical conducting wires, guidewires, and/or the like may pass. For example, the one or more electrically conductive metallic wires and the one or more polymer fibers may wrap around core element 610. Alternatively, or additionally, the one or more electrically conductive metallic wires and the one or more polymer fibers may be suspended in substrate 608. Core element 610 and/or substrate 608 may be constructed of any suitable material. In some examples, substrate 608 comprises a polymer, such as a polyether block amide, commonly known as PEBAX or Vestamide, In some examples, substrate 608 is configured to act as the polymer coating of wire 602 and/or wire 604. Alternatively, or additionally, wire 602 and/or wire 604 may have separate polymer coating (not shown in FIG. 6B) which may be a relatively high melt-temperature thermoplastic or an additional cured, cross-linked thermoset polymer which would remain stable as the lower melt-temperature thermoplastic, polyether block amide jacket is extruded over the braid. For example, the additional polymer coating may be an SI Polyimide coating.

In some examples, core 610 may be approximately 0.080 inches in diameter. In some examples, the plurality of electrically conductive metallic wires may include five pairs of wires each with a diameter of approximately 0.002 inches in diameter. In some examples, metallic element 600 may include an outer shell 612. In some examples, outer shell may have an outer diameter of approximately 0.105 inches and an inner diameter of approximately 0.095 inches.

FIG. 6C depicts a cross section of metallic element 600 after removal of a portion of substrate 608. As can be seen, wire 602 and wire 604 are now partially exposed to the surrounding environment. As such wire 602 and/or wire 604 may now serve as electrode(s), such as return electrodes.

FIG. 6D depicts wire 602 prior to removal of at least a portion of a polymer coating. In the example of FIG. 6D, wire 602 includes an electrically conductive metallic core 622 and a polymer coating 620. Polymer coating 620 may be configured to act as an insulator for wire 602, insulating electrically conductive metallic core 622 from other wires, such as wire 604 (FIGS. 6A-6C).

FIG. 6E depicts wire 602 after removal of a portion of polymer coating 620. As can be seen, a right portion of polymer coating 620 has been removed, exposing electrically conductive metallic core 622 to the surrounding environment to the right of wire 602. In some examples, rather than removing a portion of polymer coating 620, all of polymer coating 620 may be removed.

FIG. 7 is a conceptual diagram illustrating an example metallic element in accordance with one or more aspects of this disclosure. Metallic element 700 of FIG. 7 may include a plurality of electrically conductive metallic wires including a polymer coated portion 702 and an exposed metallic portion 710. Polymer coated portion 702 may include wires coated with a polymer coating as discussed herein. The polymer coating on portions of the electrically conductive metallic wires may be removed, such as through laser ablation, thermal ablation, or mechanical force, to create or form exposed metallic portion 710.

An exposed metallic portion, such as exposed metallic portion 710, may be located at a single location (e.g., at exposed metallic portion 118 of FIGS. 1A-1B) on elongated structure 112 or at a plurality of locations (e.g., under electrodes 110C, 110D, and at exposed metallic portion 118 of FIGS. 1A-1B). In some examples, as discussed above, one or more exposed metallic portions may be utilized, such as in conjunction with one or more body surface patch electrodes, to determine a location of the exposed metallic portions and associated elements of elongated structure 112 in the body of a patient.

Different techniques may be utilized to implement a system having a single exposed metallic portion than to implement a system having a plurality of exposed metallic portions, in particular for systems in which one may determine a location of the exposed metallic portion(s) in the body of the patient as discussed herein. For example, to create a single exposed metallic portion 710, one may expose all filars (or filaments) simultaneously. For example, in a typical braided elongated structure, 16 may be a typical number of filars and all 16 filars may be exposed to the laser ablation, thermal ablation, mechanical force, or the like, simultaneously. These now exposed filars may be joined (e.g., electrically coupled) to a common conductor in a handle (not shown) of system 100 that terminates at a pin in the connector that connects elongated structure 112 to conductor 114 or 116. In such an example, insulation (e.g., the polymer coating) between individual filars may not be present. For example, the polymer coating may completely (or nearly completely) removed from the portion of filars in exposed metallic portion 710 or the polymer coating on the electrically conductive metallic wires prior to creating the exposed metallic portion 710 may have had little or no polymer coating between the individual filars. For example, the polymer coating may have been deposited on the electrically conductive metallic wires while the electrically conductive metallic wires were arranged in the braid pattern such that minimal or no polymer coating is deposited between the electrically conductive metallic wires.

In some examples, a braid angle 722 relative to a horizontal axis 720 is relatively large, at least at the location of the exposed metallic portion (e.g., exposed metallic portion 118) so as to expose a relatively large surface area of metal in a relatively small amount of exposed elongated structure length along horizontal axis 720. For example, braid angle 722 may be greater than 45 degrees. An achievable braid angle will be affected by the size of the wire (e.g., a diameter for round cross-sectioned wire, width for flat wire, etc.) and the diameter of elongated structure 112. For an equivalent diameter of elongated structure 112, the larger the wire, the lower the achievable braid angle. Braid angle 722 may therefore be inherently different between different catheter designs.

Because both an acceptable signal-to-noise ratio for exposed metallic portion 710 may be highly desirable and the overall impedance created by the length and diameter of the wires of the exposed metallic portion 710 may be of concern, an Ag-cored MP35N material may be used for metallic element 700 as such a material may balancing mechanical properties of elongated structure 112 with electrical resistance. The polymer coating of polymer coated portion 702 may include LaRC-SI (or Genymer) polyimide and/or any of the polymers discussed herein.

In the case where system 100 includes a plurality of exposed metallic portions, the following may be performed. First, each of the filars may be isolated from each other with an insulation material that is sufficiently robust to abrasion at the crossover points between filars. This may be more important in any deflectable or highly flexible segments of elongated structure 112. For example, without such isolation, all exposed filars may be shorted together and a single “center of mass” point may be determined to be the location of exposed metallic portion, for example the midpoint of all exposed locations, which may be undesirable. Second, the insulation may be removed from a specific filar at a specific location. For example, transparent elongated structure or shaft jackets with individually colored insulations coupled with a vision system and laser ablation may be used to remove insulation from specific filars. Third, each exposed metallic portion 710 may include a sufficient amount of exposed metal to generate an acceptable signal-to-noise ratio, where the noise may not only come from static, but also from the environment when elongated structure 118 may be moving through a heart chamber or elsewhere in a patient.

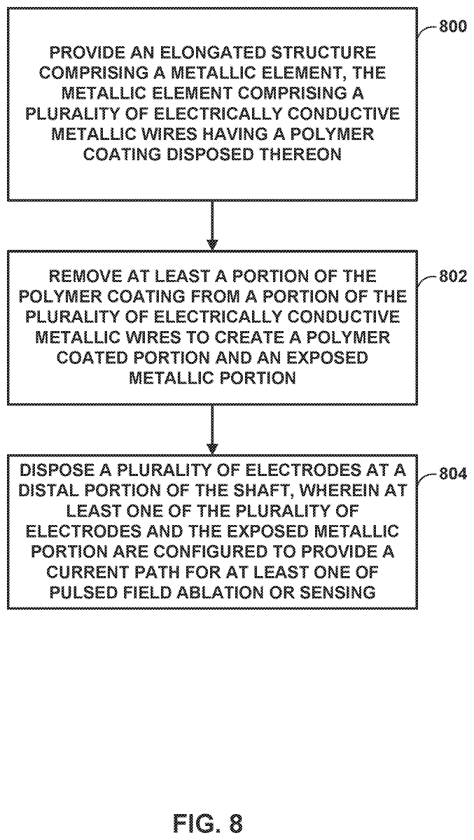

FIG. 8 is a flowchart illustrating techniques for manufacturing an ablation catheter in accordance with one or more aspects of this disclosure. A catheter manufacturer may provide an elongated structure comprising a metallic element, the metallic element comprising a plurality of electrically conductive metallic wires having a polymer coating disposed thereon (800). For example, a manufacture may manufacture or purchase an elongated structure. The elongated structure may include metallic element 600 (FIG. 6). Metallic element 600 may include a metallic braid or a plurality of metallic coils. The metallic braid or plurality of metallic coils may include a plurality of electrically conductive metallic wires, such as wire 602 and wire 604. The plurality of electrically conductive metallic wires may have a polymer coating disposed thereon.

The manufacturer may remove at least a portion of the polymer coating from a portion of the plurality of electrically conductive metallic wires to create a polymer coated portion and an exposed metallic portion (802). For example, the manufacturer may remove some or all of the polymer coating from a portion of one or more of wire 602 or wire 604 through laser ablation, thermal ablation, or mechanical force to create an exposed metallic portion, such as exposed metallic portion 118 (FIG. 1).

The manufacturer may dispose a plurality of electrodes at a distal portion of the elongated structure, wherein at least one of the plurality of electrodes and the exposed metallic portion are configured to provide a current path for at least one of pulsed field ablation or sensing (804). For example, the manufacture may crimp, swage, weld, or otherwise attach electrodes 110 (FIG. 1) at a distal portion of elongated structure 112 (FIG. 1). For example, at least one of the electrodes and the exposed metallic portion may be used to generate a pulsed field for ablation. In some examples, the exposed metallic portion may provide a return path for current out of the body of the patient to a pulsed field ablation generator.

In some examples, metallic element 600 further comprises polymer fibers interwoven among the plurality of electrically conductive metallic wires, e.g., wire 602 and wire 604. In some examples, the plurality of electrically conductive metallic wires are insulated from each other. In some examples, the plurality of electrical conductive metallic wires are not insulated from each other at least in the exposed metallic portion. In some examples, the metallic element includes a braid and wherein an angle of a braid angle from a horizontal axis of the elongated structure comprises at least 45 degrees in the exposed metallic portion. In some examples, the metallic element is configured to be adjustable so as to alter a density of the plurality of wires within the elongated structure, such as shown in FIG. IB. For example, the metallic element may be coupled to a mechanical device or lever that a clinician may use via a handle to stretch the elongated element or compact the elongated element.

In some examples, the exposed metallic portion is a first exposed metallic portion located at a first location of the elongated structure, and the manufacturer may remove at least a portion of the polymer coating from the plurality of electrically conductive metallic wires at a second location of the elongated structure to create a second exposed metallic portion configured to conduct electricity to at least one of the plurality of electrodes. The catheter manufacturer may attach (e.g., crimp, swage, weld, or otherwise attach) at least one of the plurality of electrodes onto the second exposed metallic portion.

In some examples, the catheter manufacturer may laser cut the plurality of electrodes from an electrically conductive tube into a curved shape, a pigtail shape, or a spiral shape. In some examples, the catheter manufacturer may laser cut a ribbon, the ribbon being a portion of the tube and being coupled to at least one of the plurality of electrodes. In some examples, the polymer coating includes polyimide, nylon, PTFE (polytetrafluoroethylene), FEP (fluorinated ethylene propylene), ETFE (ethylene tetrafluoroethylene), PVDF (polyvinylidene difluoride), polyurethane-nylon, crosslinked high-density polyethylene or polypropylene, Soluble Imide (SI) polyimide (formerly known as Genymer, Genymer SI), or other relatively high dielectric strength polymer.

In some examples, the catheter manufacturer may attach an expandable structure to a distal portion of the elongated structure. In some examples, the manufacturer may attach at least one of the plurality of electrodes to the expandable structure. In some examples, the expandable structure is configured to expand to move a respective location of the at least one of the plurality of electrodes with respect to target tissue in an organ of a patient. In some examples, the expandable structure comprises a balloon or a mechanical collapsible structure.

The techniques described in this disclosure may be implemented, at least in part, in hardware, software, firmware or any combination thereof. For example, various aspects of the described techniques may be implemented within processing circuitry, which may include one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also form one or more processors or processing circuitry configured to perform one or more of the techniques of this disclosure.

Such hardware, software, and firmware may be implemented, and various operation may be performed within same device, within separate devices, and/or on a coordinated basis within, among or across several devices, to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components. Processing circuitry described in this disclosure, including a processor or multiple processors, may be implemented, in various examples, as fixed-function circuits, programmable circuits, or a combination thereof. Fixed-function circuits refer to circuits that provide particular functionality with preset operations. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive stimulation parameters or output stimulation parameters), but the types of operations that the fixed-function circuits perform are generally immutable. In some examples, one or more of the units may be distinct circuit blocks (fixed-function or programmable), and in some examples, one or more of the units may be integrated circuits.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

1. An ablation catheter comprising:
an elongated structure configured to be at least partially inserted into a patient, the elongated structure comprising a metallic element, the metallic element comprising a plurality of electrically conductive metallic wires comprising a polymer coated portion and an exposed metallic portion; and
a plurality of electrodes disposed at a distal portion of the elongated structure,
wherein at least one of the plurality of electrodes and the exposed metallic portion are configured to provide a current path for at least one of pulsed field ablation or sensing.

2. The ablation catheter of claim 1, wherein the metallic element further comprises polymer fibers interwoven among the plurality of electrically conductive metallic wires.

3. The ablation catheter of claim 1, wherein the plurality of electrically conductive metallic wires are insulated from each other.

4. The ablation catheter of claim 1, wherein the plurality of electrically conductive metallic wires are not insulated from each other at least in the exposed metallic portion.

5. The ablation catheter of claim 1, wherein the metallic element comprises a braid and wherein an angle of a braid angle from a horizontal axis of the elongated structure comprises at least 45 degrees in the exposed metallic portion.

6. The ablation catheter of claim 1, wherein the metallic element is configured to be adjustable so as to alter a density of the plurality of electrically conductive metallic wires within the elongated structure.

7. The ablation catheter of claim 1, wherein the exposed metallic portion is a first exposed metallic portion at a first location of the elongated structure, and wherein the plurality of electrically conductive metallic wires comprise:
a second exposed metallic portion at a second location of the elongated structure configured to conduct electricity to at least one of the plurality of electrodes, wherein the at least one of the plurality of electrodes is electrically coupled to the second exposed metallic portion.

8. The ablation catheter of claim 1, where the plurality of electrodes are laser cut from an electrically conductive tube and are curved shaped, pigtail shaped, or spiral shaped.

9. The ablation catheter of claim 8, further comprising a ribbon, the ribbon being a portion of the electrically conductive tube and being coupled to at least one of the plurality of electrodes.

10. The ablation catheter of claim 1, further comprising an expandable structure on a distal portion of the elongated structure, wherein at least one of the plurality of electrodes is disposed on the expandable structure, and wherein the expandable structure being configured to expand to move a respective location of the at least one of the plurality of electrodes with respect to target tissue in an organ of the patient.

11. The ablation catheter of claim 10, wherein the expandable structure comprises a balloon or a mechanical collapsible structure.

12. A method comprising:
providing an elongated structure comprising a metallic element, the metallic element comprising a plurality of electrically conductive metallic wires having a polymer coating disposed thereon;
removing at least a portion of the polymer coating from a portion of the plurality of electrically conductive metallic wires to create a polymer coated portion and an exposed metallic portion; and
disposing a plurality of electrodes at a distal portion of the elongated structure,
wherein at least one of the plurality of electrodes and the exposed metallic portion are configured to provide a current path for at least one of pulsed field ablation or sensing.

13. The method of claim 12, wherein the metallic element further comprises polymer fibers interwoven among the plurality of electrically conductive metallic wires.

14. The method of claim 12, wherein the plurality of electrically conductive metallic wires are insulated from each other.

15. The method of claim 12, wherein the plurality of electrically conductive metallic wires are not insulated from each other at least in the exposed metallic portion.

16. The method of claim 12, wherein the exposed metallic portion is a first exposed metallic portion located at a first location of the elongated structure, the method further comprising:
removing at least a portion of the polymer coating from at least a portion of the plurality of electrically conductive metallic wires at a second location of the elongated structure to create a second exposed metallic portion configured to conduct electricity to at least one of the plurality of electrodes; and
attach at least one of the plurality of electrodes onto the second exposed metallic portion.

17. The method of claim 12, further comprising:
laser cutting the plurality of electrodes from an electrically conductive tube into a curved shape, a pigtail shape, or a spiral shape, wherein the electrically conductive tube comprises nitinol, copper, stainless steel, or another electrically conductive material.

18. The method of claim 17, wherein the electrically conductive tube comprises a ribbon, the ribbon being coupled to at least one of the plurality of electrodes.

19. The method of claim 12, further comprising:
attaching an expandable structure to a distal portion of the elongated structure; and
attaching at least one of the plurality of electrodes to the expandable structure,
wherein the expandable structure is configured to expand to move a respective location of the at least one of the plurality of electrodes with respect to target tissue in an organ of a patient.

20. The method of claim 19, wherein the expandable structure comprises a balloon or a mechanical collapsible structure.
